(19) United States
(12) Patent Application Publication
(10) Pub. No.: US 2026/0354793 A1
(43) Pub. Date: Nov. 12, 2026

(54) ULTRASOUND TOMOGRAPHIC IMAGE PROCESSING APPARATUS AND ULTRASOUND TOMOGRAPHIC IMAGE PROCESSING PROGRAM

(71) Applicant: FUJIFILM Corporation, Tokyo (JP)

(72) Inventor: Tomoaki CHONO, Tokyo (JP)

(73) Assignee: FUJIFILM Corporation, Tokyo (JP)

(21) Appl. No.: 19/654,423

(22) Filed: Apr. 21, 2026

(30) Foreign Application Priority Data
May 7, 2025 (JP) 2025-077170

(51) Int. Cl. A61B 8/13; A61B 8/00; A61B 8/08

(52) U.S. Cl. CPC A61B 8/13; A61B 8/0883; A61B 8/461; A61B 8/5215

(57) ABSTRACT

A tracking processing unit executes tracking processing in an ultrasound tomographic image sequence representing a periodic motion of a heart that is a target tissue, to calculate a strain for each of a plurality of regions of the heart. A reliability evaluation unit calculates a reliability evaluation value representing reliability of the tracking processing for each region. An adoption/rejection determination unit determines a region in which the reliability evaluation value is less than an evaluation value threshold as a non-target region. A display controller displays the non-target region on a display to notify an operator of the non-target regions. In addition, a global strain calculation unit calculates a global strain without using the strain of the non-target region.

### CROSS-REFERENCE TO RELATED APPLICATION

This application claims the priority benefit of Japan application serial no. 2025-077170, filed on May 7, 2025. The entirety of the above-mentioned patent application is hereby incorporated by reference herein and made a part of this specification.

### BACKGROUND OF THE INVENTION

### 1. Field of the Invention

The present specification discloses improvements in an ultrasound tomographic image processing apparatus and an ultrasound tomographic image processing program.

### 2. Description of the Related Art

In the related art, an ultrasound diagnostic apparatus that forms an ultrasound tomographic image representing a cross section of a subject by transmitting and receiving ultrasound waves to and from the subject is known. In particular, in the related art, a deformation amount in a periodic motion of a target tissue (for example, a heart or a blood vessel) is calculated using an ultrasound tomographic image sequence consisting of a plurality of ultrasound tomographic images arranged in time series, which represent the target tissue performing the periodic motion. For example, in a case where the target tissue is a heart, strain analysis is performed in which a deformation amount (strain) indicating how much the heart is contracted from end-diastole to end-systole is analyzed using an ultrasound tomographic image sequence consisting of ultrasound tomographic images arranged in time series, which represent the heart.

In order to calculate the deformation amount in the periodic motion of the target tissue, tracking processing, which is processing of tracking the target tissue over time in the ultrasound tomographic image sequence, is required. For example, in strain analysis of a heart, a speckle tracking method of tracking small speckles (speckle) of a myocardium on an ultrasound tomographic image is used.

Reliability (in other words, accuracy) of the tracking processing of the target tissue greatly affects a calculation result of the deformation amount in the periodic motion of the target tissue. Therefore, in the related art, the reliability of the tracking processing of the target tissue is evaluated, and the user is notified of the reliability.

For example, JP2007-296336A discloses an ultrasound system that tracks a heart to measure cardiac activity, the ultrasound system dividing the heart into a plurality of divided regions and displaying a tracking quality indicator for a region having a tracking quality lower than a predetermined level.

In addition, JP2019-141380A discloses an ultrasound signal processing apparatus that evaluates reliability of a result of velocity vector calculation of motion of a target tissue, the ultrasound signal processing apparatus approximately calculating a parallel-shifted signal generated by parallel-shifting an echo signal of a predetermined temporal phase obtained by transmitting and receiving ultrasound waves to and from a subject at a predetermined time and a predetermined speed, calculating a difference between the parallel-shifted signal and an actual echo signal after the predetermined time as a deviation signal, extracting an image deformation component, which is a change component of a signal value due to speckles or the like, from the deviation signal based on the deviation signal, the parallel-shifted signal, and the actual echo signal after the predetermined time, calculating an error energy due to deformation of an image such as speckles based on the image deformation component, the deviation signal, the parallel-shifted signal, and the actual echo signal after the predetermined time, and displaying a calculation result of the error energy.

### SUMMARY OF THE INVENTION

Meanwhile, in some cases, the target tissue is conceptually divided into a plurality of predetermined regions, and then a deformation amount in a periodic motion of the target tissue is calculated for each region. For example, in strain analysis of a heart, the heart (myocardium) is conceptually divided into a plurality of regions, and then strain is calculated for each region. Accordingly, the user can easily ascertain the strain for each region of the heart.

Further, an overall deformation amount of the target tissue as a whole may be calculated based on the deformation amount for each of the plurality of regions. For example, in the strain analysis of the heart, global strain such as global longitudinal strain (GLS), global radial strain (GRS), and global circumferential strain (GCS) is calculated as the overall deformation amount. The global strain can be a representative value (for example, an average value) of the strain of each region.

Here, in a case where reliability of the tracking processing is low in any of the plurality of regions, the deformation amount of the region may not be accurate. In such a case, in a case where the overall deformation amount is calculated including the deformation amount of the region, the overall deformation amount is also not accurate. Therefore, it is desirable to calculate the overall deformation amount by excluding the deformation amount of the region. In addition, in a case where the overall deformation amount is calculated by excluding the deformation amount of the region having low reliability of the tracking processing, the user may be able to easily ascertain which region's deformation amount is excluded in a case where the overall deformation amount is calculated.

An object of an ultrasound tomographic image processing apparatus disclosed in the present specification is to enable a user to easily ascertain a region in which a deformation amount is not used for calculating an overall deformation amount among a plurality of regions of a target region that performs periodic motion. Alternatively, an object of the ultrasound tomographic image processing apparatus disclosed in the present specification is to suppress a decrease in calculation accuracy of an overall deformation amount of a target tissue that performs periodic motion without requiring a user to take time and effort.

An ultrasound tomographic image processing apparatus disclosed in the present specification comprises: a processor, in which the processor is configured to: acquire an ultrasound tomographic image sequence that is formed by transmitting and receiving an ultrasonic wave to and from a target tissue of a subject and that is arranged in a time series representing a periodic motion of the target tissue; calculate a deformation amount of the target tissue in the periodic motion for each of a plurality of regions of the target tissue defined in advance by executing tracking processing for tracking each part of the target tissue over time in the ultrasound tomographic image sequence; calculate a reliability evaluation value representing reliability of the tracking processing for each of the regions; determine regions in which the reliability evaluation value is less than a predetermined evaluation value threshold, as non-target regions in which the deformation amount is not used in a case of calculating an overall deformation amount that is a deformation amount of the target tissue as a whole in the periodic motion, based on the deformation amount for each of the regions; and notify a user of the non-target regions.

The processor may be configured to display, on a display unit, a target region that is a region in which the deformation amount is used in a case of calculating the overall deformation amount and the non-target region in a model diagram of the target tissue in which the plurality of regions are shown, in an identifiable manner.

The processor may be configured to display at least one ultrasound tomographic image included in the ultrasound tomographic image sequence on a display unit, display, on the display unit, a plurality of superimposed images that are superimposed on each of the regions on the ultrasound tomographic image to represent each of the regions and that correspond to each of the regions, and display the superimposed image corresponding to the target region that is the region in which the deformation amount is used in a case of calculating the overall deformation amount and the superimposed image corresponding to the non-target region in an identifiable manner.

The processor may be configured to display, on a display unit, a difference between the overall deformation amount calculated by using the deformation amount of each of the regions including the non-target region and the overall deformation amount calculated without using the deformation amount of the non-target region.

The processor may be configured to notify the user that the overall deformation amount is not calculated in a case in which the number of non-target regions in the plurality of regions is equal to or greater than a predetermined number.

In addition, an ultrasound tomographic image processing apparatus disclosed in the present specification comprises: a processor, in which the processor is configured to: acquire an ultrasound tomographic image sequence that is formed by transmitting and receiving an ultrasonic wave to and from a target tissue of a subject and that is arranged in a time series representing a periodic motion of the target tissue; calculate a deformation amount of the target tissue in the periodic motion for each of a plurality of regions of the target tissue defined in advance by executing tracking processing for tracking each part of the target tissue over time in the ultrasound tomographic image sequence; calculate a reliability evaluation value representing reliability of the tracking processing for each of the regions; calculate an overall deformation amount that is a deformation amount of the target tissue as a whole in the periodic motion, based on the deformation amount of regions in which the reliability evaluation value is equal to or greater than an evaluation value threshold, among the plurality of regions; and display the overall deformation amount on a display unit in a case in which the overall deformation amount is calculated.

The processor may be configured not to calculate the overall deformation amount in a case in which the number of the regions in which the reliability evaluation value is less than the evaluation value threshold is equal to or greater than a predetermined number, in the plurality of regions.

In addition, an ultrasound tomographic image processing program disclosed in the present specification causes a computer to execute a process including: acquiring an ultrasound tomographic image sequence that is formed by transmitting and receiving an ultrasonic wave to and from a target tissue of a subject and that is arranged in a time series representing a periodic motion of the target tissue; calculating a deformation amount of the target tissue in the periodic motion for each of a plurality of regions of the target tissue defined in advance by executing tracking processing for tracking each part of the target tissue over time in the ultrasound tomographic image sequence; calculating a reliability evaluation value representing reliability of the tracking processing for each of the regions; determining regions in which the reliability evaluation value is less than a predetermined evaluation value threshold, as non-target regions in which the deformation amount is not used in a case of calculating an overall deformation amount that is a deformation amount of the target tissue as a whole in the periodic motion, based on the deformation amount for each of the regions; and notifying a user of the non-target regions.

In addition, an ultrasound tomographic image processing program disclosed in the present specification causes a computer to execute a process including: acquiring an ultrasound tomographic image sequence that is formed by transmitting and receiving an ultrasonic wave to and from a target tissue of a subject and that is arranged in a time series representing a periodic motion of the target tissue; calculating a deformation amount of the target tissue in the periodic motion for each of a plurality of regions of the target tissue defined in advance by executing tracking processing for tracking each part of the target tissue over time in the ultrasound tomographic image sequence; calculating a reliability evaluation value representing reliability of the tracking processing for each of the regions; calculating an overall deformation amount that is a deformation amount of the target tissue as a whole in the periodic motion, based on the deformation amount of regions in which the reliability evaluation value is equal to or greater than an evaluation value threshold, among the plurality of regions; and displaying the overall deformation amount on a display unit in a case in which the overall deformation amount is calculated.

With the ultrasound tomographic image processing apparatus disclosed in the present specification, the user can easily ascertain a region in which a deformation amount is not used for calculating an overall deformation amount among a plurality of regions of a target region that is in periodic motion. Alternatively, with the ultrasound tomographic image processing apparatus disclosed in the present specification, it is possible to suppress a decrease in calculation accuracy of an overall deformation amount of a target tissue that is in periodic motion without causing a burden on the user.

### BRIEF DESCRIPTION OF THE DRAWINGS

FIG. 1 is a schematic diagram of a configuration of an ultrasound diagnostic apparatus according to the present embodiment.

FIG. 2 is a diagram showing an example of an ultrasound tomographic image included in an ultrasound tomographic image sequence.

FIG. 3 is a diagram showing an example of a tracking point.

FIG. 4 is a conceptual diagram showing a plurality of regions of a heart.

FIG. 5 is a diagram showing an example of a notification of a result of tracking processing, global strain, and reliability evaluation.

FIG. 6 is a first diagram showing a display example of a non-target region.

FIG. 7 is a second diagram showing a display example of a non-target region.

FIG. 8 is a diagram showing a display example of a case where global strain is not calculated.

FIG. 9 is a flowchart showing a flow of a process of the ultrasound diagnostic apparatus according to the present embodiment.

### DESCRIPTION OF THE PREFERRED EMBODIMENTS

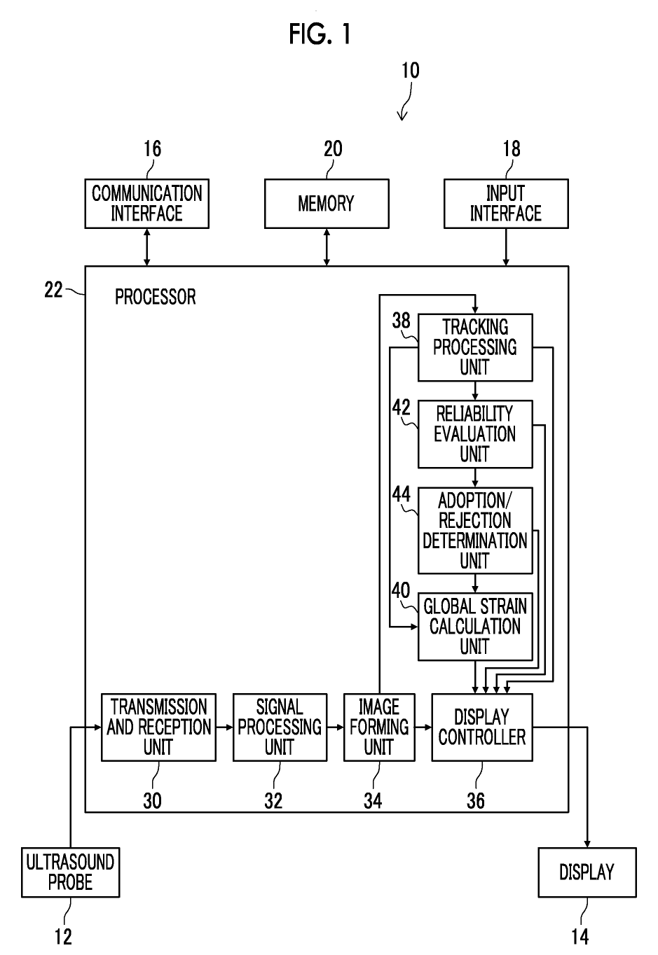

FIG. 1 is a schematic diagram showing a configuration of an ultrasound diagnostic apparatus 10 as an ultrasound tomographic image processing apparatus according to the present embodiment. The ultrasound diagnostic apparatus 10 is a medical apparatus installed in a medical institution such as a hospital.

An ultrasound probe 12 includes a vibration element array including a plurality of vibration elements. A transmission signal, which is an electric signal, is supplied from a processor 22, which will be described below, to the vibration element array, so that ultrasound waves are transmitted toward a subject. In addition, the vibration element array receives reflected waves of the transmitted ultrasound waves from the subject and supplies a reception signal, which is an electric signal, to the processor 22.

In the present embodiment, the ultrasound probe 12 transmits and receives ultrasound waves to and from a target tissue of the subject. The target tissue is a tissue that performs a periodic motion (cyclic motion), and is, for example, a heart or a blood vessel. In the present embodiment, an example in which the target tissue is a heart will be described. In particular, the ultrasound probe 12 transmits the ultrasound waves to the target tissue over at least one cycle (preferably, a plurality of cycles) in the periodic motion.

The display 14 as a display unit is a display device configured by, for example, a liquid crystal display or an organic electro luminescence (EL). Various screens are displayed on the display 14 in response to an instruction from the processor 22.

The communication interface 16 is configured as, for example, a network adapter. The communication interface 16 exhibits a function of communicating with another device via a communication line such as a local area network (LAN) or a wide area network (WAN).

An input interface 18 is configured by, for example, a button, a trackball, a touch panel, or the like. The input interface 18 is used to input an instruction of an operator of the ultrasound diagnostic apparatus 10 as a user of the ultrasound tomographic image processing apparatus to the ultrasound diagnostic apparatus 10.

A memory 20 includes a hard disk drive (HDD), a solid state drive (SSD), an embedded multi media card (eMMC), a read only memory (ROM), a random access memory (RAM), or the like. The memory 20 stores the ultrasound tomographic image processing program for operating each unit of the ultrasound diagnostic apparatus 10. In addition, the ultrasound tomographic image processing program can also be stored, for example, in a non-transitory computer-readable storage medium such as a universal serial bus (USB) memory or a CD-ROM. The ultrasound diagnostic apparatus 10 can read the ultrasound tomographic image processing program from the storage medium and execute the ultrasound tomographic image processing program.

A processor 22 is configured to include, for example, a central processing unit (CPU). The processor 22 exhibits functions of each part shown in FIG. 1 by an ultrasound diagnosis support program stored in the memory 20.

A transmission and reception unit 30 transmits a transmission signal to the ultrasound probe 12 (specifically, each vibration element of the vibration element array). As a result, the ultrasound probe 12 transmits the ultrasound wave to the target tissue. In addition, the transmission and reception unit 30 receives a reception signal from each vibration element that has received a reflected wave of the ultrasound wave from the target tissue. The transmission and reception unit 30 has an adder and a plurality of delay elements corresponding to the respective vibration elements, and performs phase addition processing of aligning and adding phases of reception signals from the respective vibration elements by the adder and the plurality of delay elements. As a result, a reception beam signal in which information indicating the signal intensity of the reflected waves from the subject is arranged in a depth direction of the subject is formed.

A signal processing unit 32 performs various types of signal processing including filter processing of applying a bandpass filter, detection processing, and the like, on the reception beam signal from the transmission and reception unit 30.

An image forming unit 34 forms an ultrasound tomographic image (B-mode image) representing a cross section of the target tissue based on the reception beam signal subjected to the signal processing in the signal processing unit 32. In particular, the image forming unit 34 forms an ultrasound tomographic image sequence consisting of a plurality of ultrasound tomographic images arranged in time series representing the periodic motion of the target tissue. In other words, the image forming unit 34 forms an ultrasound tomographic image sequence representing a cross section of the target tissue in each temporal phase of the periodic motion of the target tissue.

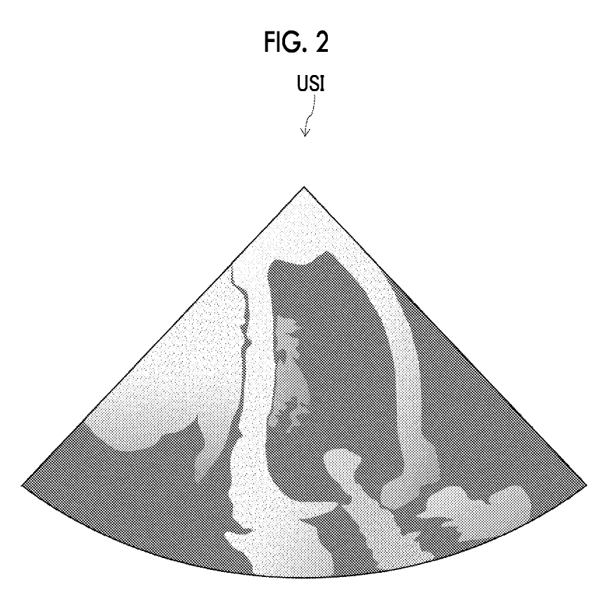

FIG. 2 is a diagram showing an example of an ultrasound tomographic image USI included in an ultrasound tomographic image sequence. In the present embodiment, since the target tissue is the heart, the ultrasound tomographic image USI represents a cross section of the heart. In the present embodiment, in particular, since a longitudinal strain (LS) that is a strain representing expansion and contraction of the heart in a major axis direction is analyzed, the ultrasound tomographic image USI shows a major axis cross section of the heart. It should be noted that, as a type of the strain of the heart, in addition to the LS, there is a radial strain (RS) representing contraction of the heart in a central portion direction or a circumferential strain (CS) representing contraction of the heart in a circumferential direction. The present invention can be suitably applied not only to the LS but also to a case of performing the strain analysis related to the RS or the CS.

The ultrasound tomographic image sequence formed by the image forming unit 34 is stored in a cine memory (not shown).

The display controller 36 performs control of displaying the ultrasound tomographic image formed by the image forming unit 34 on a display 14. In addition, the display controller 36 performs control of displaying various types of information indicating processing results of a tracking processing unit 38, a global strain calculation unit 40, a reliability evaluation unit 42, and an adoption/rejection determination unit 44, which will be described below, on the display 14.

The tracking processing unit 38 executes tracking processing of tracking each part of the target tissue over time in the ultrasound tomographic image sequence formed by the image forming unit 34.

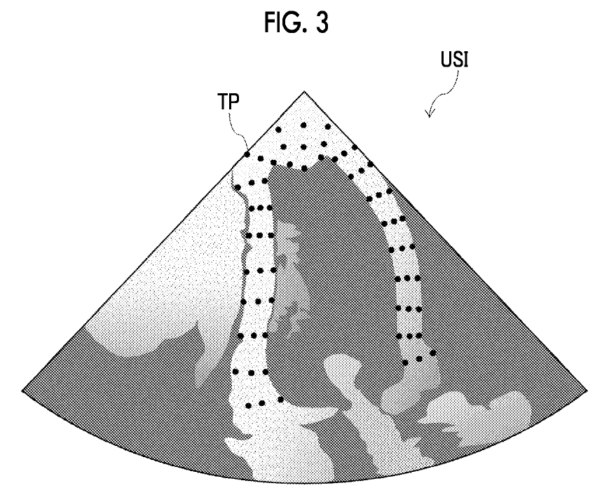

FIG. 3 is a diagram showing an example of a tracking point TP. The tracking processing unit 38 sets a plurality of tracking points TP at positions of a plurality of speckles in the myocardium by analyzing the ultrasound tomographic images USI included in the ultrasound tomographic image sequence. The tracking processing unit 38 may set the tracking point TP in response to an instruction from the operator.

Next, the tracking processing unit 38 specifies the position of the speckle corresponding to each tracking point TP in another ultrasound tomographic image that is temporally adjacent to the ultrasound tomographic image USI in which the plurality of tracking points TP are set, the ultrasound tomographic image USI being included in the ultrasound tomographic image sequence. Since a known method can be used as a method of specifying the position, a detailed description thereof will be omitted here. As a result, a movement vector is specified with the position of the tracking point TP specified in a certain ultrasound tomographic image as a start point and the position of the tracking point TP specified in another ultrasound tomographic image that is temporally adjacent to the ultrasound tomographic image as an end point. The position of each speckle (the position of each tracking point TP) in the ultrasound tomographic image sequence is tracked by specifying the movement vector for each tracking point TP between the ultrasound tomographic images that are temporally adjacent to each other in the ultrasound tomographic image sequence. That is, the position of the heart (myocardium) in the ultrasound tomographic image sequence is tracked over time.

The tracking processing unit 38 calculates the deformation amount (strain) of the heart based on the result of the tracking processing in the ultrasound tomographic image sequence for one cycle of the heart. The strain of the heart is an indicator indicating how much the heart is contracted with respect to the end-diastole of the heart in the end-systole of the heart. The strain is represented as, for example, "-20%".

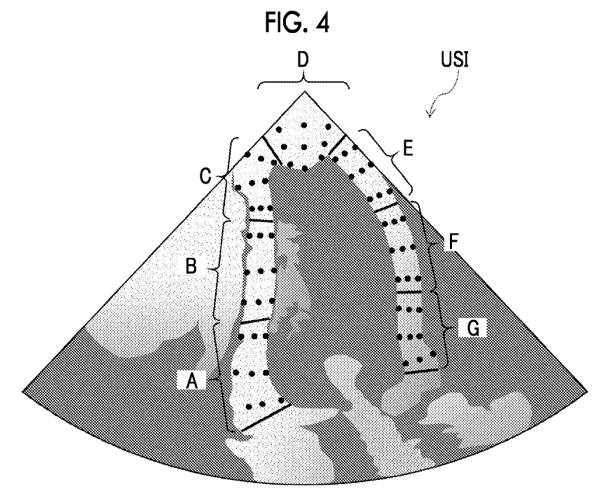

FIG. 4 is a conceptual diagram showing a plurality of regions of the heart. As shown in FIG. 4, in the ultrasound tomographic image USI, the heart (myocardium) is conceptually divided into a plurality of regions A to G in advance. The tracking processing unit 38 calculates the deformation amount (strain) of the heart in the periodic motion for each of the plurality of regions of the heart defined in advance.

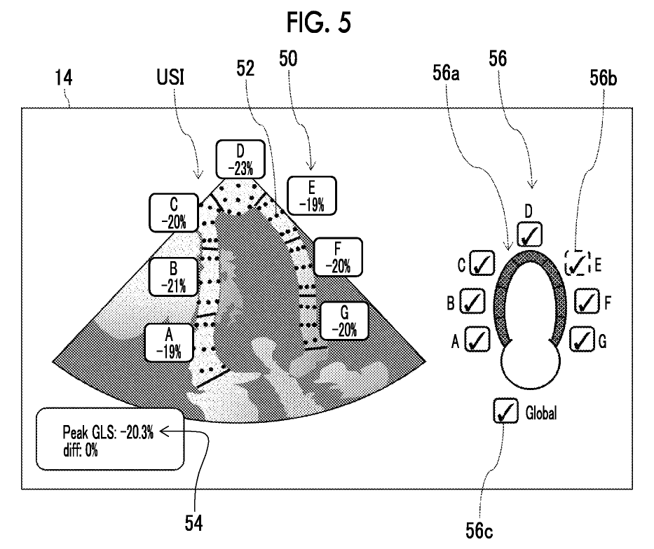

FIG. 5 is a diagram showing a notification example of the result of the tracking processing. The display controller 36 displays the result of the tracking processing by the tracking processing unit 38 on the display 14. Specifically, the display controller 36 displays the strain 50 calculated for each region of the heart on the display 14. As a result, the operator can easily ascertain the strain for each region.

As shown in FIG. 5, the display controller 36 may display the ultrasound tomographic image USI (which may be a moving image) included in the ultrasound tomographic image sequence on the display 14, and further display the strain 50 of each region after displaying each region of the heart on the ultrasound tomographic image USI. As a method of displaying each region of the heart on the ultrasound tomographic image USI, for example, each region may be displayed by superimposing the region on the ultrasound tomographic image USI, and a plurality of superimposed images corresponding to each region may be displayed. In the example of FIG. 5, as such a superimposed image, the display controller 36 displays a tracking point image 52 representing each tracking point TP. The tracking point images 52 included in the regions different from each other may have different display modes. For example, the tracking point image 52 included in the region A is displayed in green, the tracking point image 52 included in the region B is displayed in blue, and the tracking point image 52 included in the region G is displayed in red.

The global strain calculation unit 40 calculates a global strain, which is an overall deformation amount of the entire heart in the periodic motion of the heart, based on the strains of the plurality of regions of the heart calculated by the tracking processing unit 38. The global strain calculation unit 40 can calculate a representative value of the strains of the plurality of regions of the heart as the global strain. In the present embodiment, the global strain calculation unit 40 calculates an average value of the strains of the plurality of regions of the heart as the global strain.

The global strain calculation unit 40 may calculate the global strain by excluding the strains of some regions instead of the strains of all the regions of the heart. Specifically, the global strain calculation unit 40 may calculate the global strain by excluding (ignoring) the strains of some regions based on processing of a reliability evaluation unit 42 and an adoption/rejection determination unit 44, which will be described below. Details of this processing will be described below.

As shown in FIG. 5, the display controller 36 displays a global strain 54 calculated by the global strain calculation unit 40 on the display 14. As a result, the operator can easily ascertain the strain of the entire heart.

The reliability evaluation unit 42 calculates a reliability evaluation value representing the reliability of the tracking processing by the tracking processing unit 38. In the present embodiment, since the strain of the heart is calculated by the tracking processing, it can be said that the reliability evaluation value represents the reliability of the calculated strain. In particular, since the tracking processing unit 38 calculates the strain for each of the plurality of regions of the heart, the reliability evaluation unit 42 calculates the reliability evaluation value for each region. As will be described in detail below, the reliability evaluation unit 42 calculates the reliability evaluation value for each tracking point TP in each of the temporally adjacent ultrasound tomographic images in the ultrasound tomographic image sequence, summarizes the reliability evaluation value for each tracking point TP for each region of the heart in each of the ultrasound tomographic images, and further summarizes the plurality of reliability evaluation values between the ultrasound tomographic images over time in each region to calculate the reliability evaluation value for each region as the entire ultrasound tomographic image sequence (for example, in the unit period of the heart).

Various methods can be considered as a method of calculating the reliability evaluation value, but the reliability evaluation unit 42 calculates the reliability evaluation value by the following method.

For example, in a case where the tracking processing unit 38 specifies a movement vector of each tracking point TP between the temporally adjacent ultrasound tomographic images of the ultrasound tomographic image sequence, the reliability evaluation unit 42 performs the matching processing between the ultrasound tomographic images and calculates a correlation coefficient between the two images. Specifically, the correlation coefficient between the minute regions including the tracking point TP between the two images is calculated. Since the minute region does not change much between the temporally adjacent ultrasound tomographic images in many cases, the reliability evaluation unit 42 can determine that the reliability evaluation value for the minute region (including the tracking point TP) is high in a case where the correlation coefficient is large. On the contrary, in a case where the correlation coefficient is small, the reliability evaluation unit 42 can determine that the reliability evaluation value for the minute region (including the tracking point TP) is low. In this way, the reliability evaluation unit 42 can calculate the reliability evaluation value for each tracking point TP in each of the temporally adjacent ultrasound tomographic images.

In addition, as another method, the reliability evaluation unit 42 can calculate the reliability evaluation value for each tracking point TP using a feature point detection algorithm such as Harris, FAST, or SIFT. Specifically, in a case where a feature point (for example, an edge) is detected by the feature point detection algorithm, it can be said that the tracking processing is easy. Therefore, in the vicinity of each tracking point TP, as the feature point is more clearly detected by the feature point detection algorithm, it can be determined that the reliability evaluation value for the tracking point TP is high.

The reliability evaluation unit 42 summarizes the reliability evaluation value for each tracking point TP for each region of the heart. Summarizing means determining a value representing the reliability evaluation value for a plurality of tracking points TP included in the region. The reliability evaluation unit 42 calculates a representative value (for example, an average value or a peak value) of the reliability evaluation values for the plurality of tracking points TP included in the region as a reliability evaluation value summarized for the region. As a result, the reliability evaluation value is calculated for each region of the heart in each of the ultrasound tomographic images that are temporally adjacent in the ultrasound tomographic image sequence.

Further, the reliability evaluation unit 42 summarizes, for each region, the plurality of reliability evaluation values between the ultrasound tomographic images over time of the heart. Here, the reliability evaluation unit 42 calculates a representative value (for example, an average value or a peak value) of the plurality of reliability evaluation values between the ultrasound tomographic images related to a certain region as the reliability evaluation value of the region. As a result, the reliability evaluation value for each region (for example, in the unit period of the heart) as the entire ultrasound tomographic image sequence is calculated.

In a case in which the reliability evaluation value for each region of the heart is calculated as described above, the reliability evaluation unit 42 can specify a region in which the reliability evaluation value is low in the plurality of regions. Specifically, the reliability evaluation unit 42 can determine in advance an evaluation value threshold and can specify whether or not the reliability evaluation value of each region is less than the evaluation value threshold.

The display controller 36 may notify the operator of a region in which the reliability evaluation value is less than the evaluation value threshold among the plurality of regions of the heart. For example, the display controller 36 displays a schema diagram 56, which is a model diagram of the major axis cross section of the heart, on the display 14. The schema diagram 56 according to the present embodiment has a figure model 56a representing a shape of the major axis cross section of the heart. In the figure model 56a, a plurality of regions of the heart are shown. In addition, the schema diagram 56 has a region check box 56b corresponding to each region. In the present embodiment, the display controller 36 notifies the operator of a region in which the reliability evaluation value is less than the evaluation value threshold by making display modes of the region check box 56b corresponding to a region in which the reliability evaluation value is equal to or greater than the evaluation value threshold and the region check box 56b corresponding to a region in which the reliability evaluation value is less than the evaluation value threshold different from each other. In the example of FIG. 5, the display controller 36 displays the region check box 56b corresponding to a region in which the reliability evaluation value is equal to or greater than the evaluation value threshold with a solid line, and displays the region check box 56b corresponding to a region in which the reliability evaluation value is less than the evaluation value threshold with a broken line. Of course, the difference in the display mode is not limited to this, and, for example, colors or the like may be made different from each other.

The adoption/rejection determination unit 44 determines whether or not to use the strain for each region in a case of calculating the global strain based on the reliability evaluation value for each region of the heart calculated by the reliability evaluation unit 42. Specifically, the adoption/rejection determination unit 44 determines a region in which the reliability evaluation value is equal to or greater than the evaluation value threshold as a target region in which the strain is used in a case of calculating the global strain, and determines a region in which the reliability evaluation value is less than the evaluation value threshold as a non-target region in which the strain is not used in a case of calculating the global strain.

In a case where there is an instruction from the operator, the adoption/rejection determination unit 44 may set a region related to the instruction as a non-target region.

The display controller 36 notifies the operator by displaying the non-target region determined by the adoption/rejection determination unit 44 on the display 14. As a result, the operator can easily ascertain a region (that is, a non-target region) in which the strain is not used in a case of calculating the global strain among the plurality of regions of the heart.

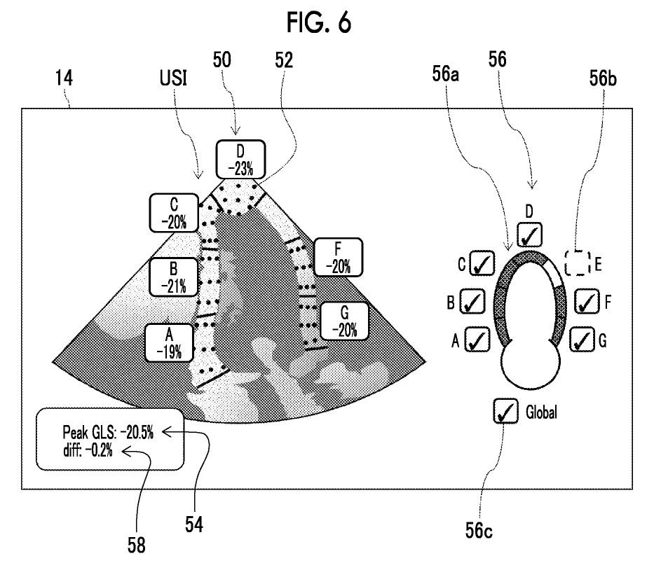

FIG. 6 is a first diagram showing a display example of a non-target region. For example, the display controller 36 displays the target region and the non-target region in an identifiable manner depending on the presence or absence of a check of a region check box 56b included in a schema diagram 56. Specifically, the display controller 36 displays a check in the region check box 56b corresponding to the target region, and does not display a check in the region check box 56b corresponding to a region E that is a non-target region.

The display controller 36 may display the target region and the non-target region in an identifiable manner in a figure model 56a of the schema diagram 56. For example, as shown in FIG. 6, the display controller 36 colors the target region in the figure model 56a, and does not color the region E that is a non-target region.

The display controller 36 may display the target region and the non-target region in an identifiable manner by a tracking point image 52 that is a superimposed image displayed on the ultrasound tomographic image USI in a superimposed manner. Specifically, the display controller 36 may display the tracking point image 52 corresponding to the target region and the tracking point image 52 corresponding to the non-target region in an identifiable manner. In the example of FIG. 6, the display controller 36 displays the tracking point image 52 corresponding to the target region, but does not display the tracking point image 52 corresponding to the region E that is a non-target region.

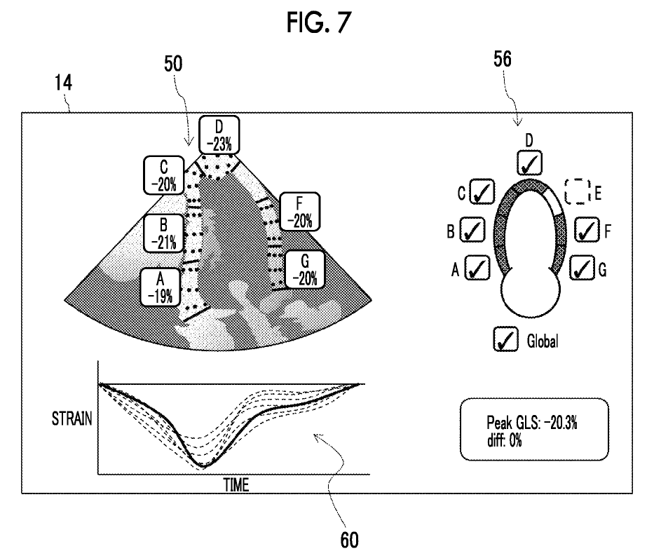

FIG. 7 is a second diagram showing a display example of a non-target region. As shown in FIG. 7, the display controller 36 may display a graph 60 showing a time change of the strain in the region of the heart together with the ultrasound tomographic image USI and the schema diagram 56. A horizontal axis of the graph 60 represents time, and a vertical axis represents the strain. In the graph 60, a time change of a global strain is indicated by a thick line, and a time change of the strain in each region is indicated by a broken line. The display controller 36 may display the target region and the non-target region in an identifiable manner in the graph 60. For example, the display controller 36 displays a broken line corresponding to the target region in the graph 60, but does not display a broken line corresponding to the non-target region.

Note that the method of notifying of the non-target region is not limited to the display on the display 14. The processor 22 may notify of the non-target region by using a sound or light (for example, turning on/off a light emitting unit corresponding to each region prepared in advance) instead of or in addition to the display on the display 14.

The global strain calculation unit 40 calculates the global strain based on the strain in the target region (that is, a region in which the reliability evaluation value is equal to or greater than the evaluation value threshold) among the plurality of regions of the heart in accordance with the determination of the adoption/rejection determination unit 44. In other words, the global strain calculation unit 40 calculates the global strain without using the strain in the non-target region (that is, a region in which the reliability evaluation value is less than the evaluation value threshold) in accordance with the determination of the adoption/rejection determination unit 44.

The non-target region in which the reliability evaluation value is less than the evaluation value threshold may have low reliability of the tracking processing, that is, the strain calculated for the non-target region may not be accurate. Therefore, in a case in which the global strain is calculated including the non-target region, the global strain may also not be accurate. Therefore, in the present embodiment, the global strain is calculated by excluding the non-target region, so that a decrease in calculation accuracy of the global strain is suppressed even in a case in which the reliability of the tracking processing of a part of the regions is low.

In a case in which the global strain is calculated without using the strain in the non-target region, the global strain calculation unit 40 may calculate a difference between the global strain calculated by using the strain in each region (that is, all the regions) including the non-target region and the global strain calculated without using the strain in the non-target region (in other words, by using only the strain in the target region).

As described above, the display controller 36 displays the global strain 54 (see FIGS. 5 or 6), but in a case in which the global strain calculation unit 40 calculates the global strain without using the strain in the non-target region, the display controller 36 may display a difference 58 between the global strain calculated by using the strain in each region including the non-target region and the global strain calculated without using the strain in the non-target region on the display 14. As a result, the operator can easily understand the difference 58, that is, the effect of calculating the global strain by excluding the non-target region.

As described above, the region in which the reliability evaluation value is less than the evaluation value threshold is the non-target region, and the strain in the non-target region is excluded to calculate the global strain, but a case in which there are a plurality of non-target regions is also considered. Here, in a case in which the number of non-target regions is equal to or greater than a predetermined number, the global strain is calculated based on the strain in a small number of regions, and the significance or credibility of the global strain may be weakened.

Therefore, in the present embodiment, in a case in which there are a predetermined number or more of non-target regions in the plurality of regions of the heart, the global strain calculation unit 40 does not calculate the global strain. In a case in which there are a predetermined number or more of non-target regions, the display controller 36 may notify the user that the global strain is not calculated. In the present embodiment, the predetermined number is set to two. That is, in a case in which there are two or more non-target regions, the global strain calculation unit 40 does not calculate the global strain.

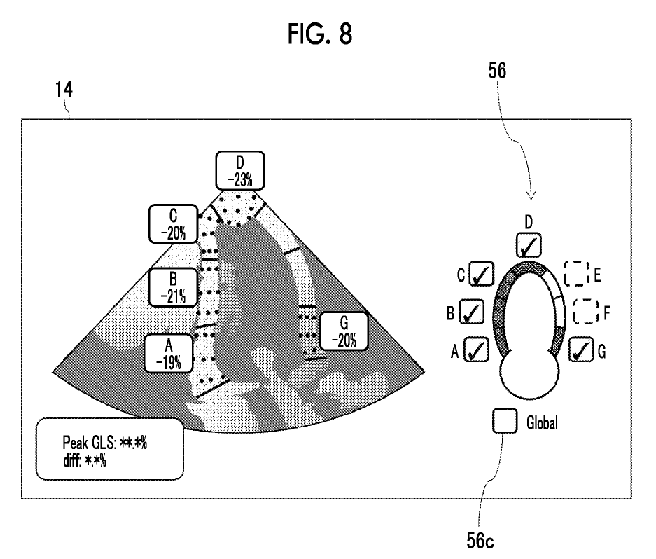

FIG. 8 is a diagram showing a display example of a case where global strain is not calculated. In the present embodiment, the schema diagram 56 further includes a global check box 56c. In a case in which the global strain is calculated by the global strain calculation unit 40, the display controller 36 displays a check in the global check box 56c (see FIGS. 5 and 6). On the other hand, in a case in which the global strain is not calculated by the global strain calculation unit 40, the display controller 36 does not display a check in the global check box 56c (see FIG. 8).

A notification method of the fact that the global strain is not calculated is not limited to the display on the display 14. The processor 22 may notify that the global strain is not calculated by using sound or light, instead of or in addition to the display on the display 14.

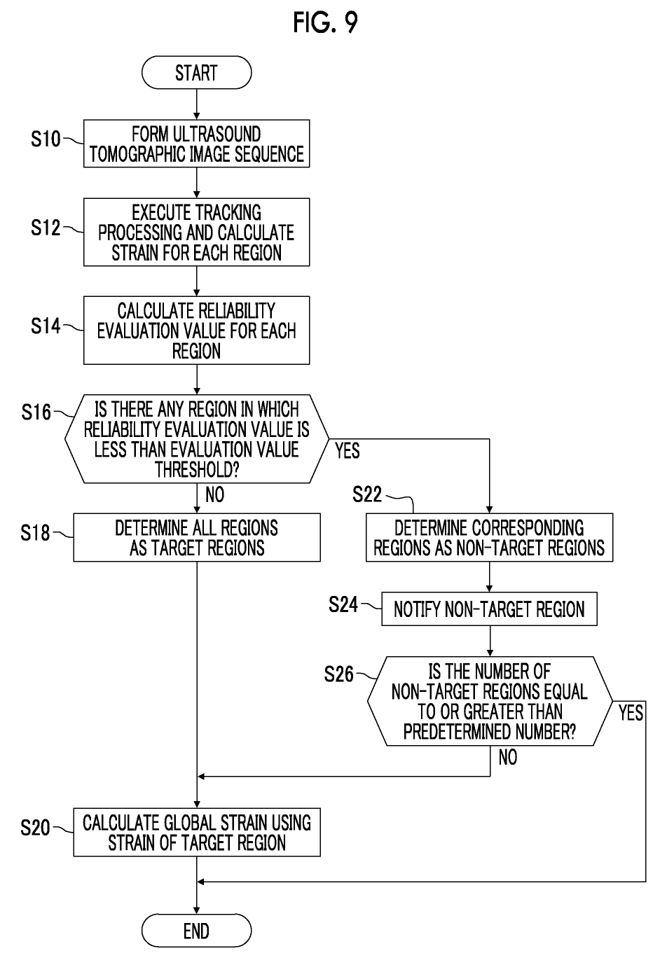

The outline of the ultrasound diagnostic apparatus 10 according to the present embodiment is described above. Hereinafter, a flow of the processing of the ultrasound diagnostic apparatus 10 will be described with reference to the flowchart shown in FIG. 9.

In step S10, the ultrasound probe 12 transmits and receives the ultrasonic waves to and from the heart as the target tissue. The image forming unit 34 forms an ultrasound tomographic image sequence showing the periodic motion of the heart based on the obtained reception signal.

In step S12, the tracking processing unit 38 executes the tracking processing of tracking each part of the heart over time in the ultrasound tomographic image sequence formed in step S10. In addition, the tracking processing unit 38 calculates the strain for each region of the heart based on a result of the tracking processing.

In step S14, the reliability evaluation unit 42 calculates the reliability evaluation value indicating the reliability of the tracking processing in step S12 for each region.

In step S16, the adoption/rejection determination unit 44 determines whether or not there is a region in which the reliability evaluation value calculated in step S14 is less than the evaluation value threshold among the plurality of regions of the heart. In a case in which the reliability evaluation value of all the regions is equal to or greater than the evaluation value threshold, the processing proceeds to step S18.

In step S18, the adoption/rejection determination unit 44 determines all the regions as the target regions used for calculating the global strain in a case in which the global strain is calculated.

In step S20, the global strain calculation unit 40 calculates the global strain using the strain of the target region. The display controller 36 displays the calculated global strain on the display 14.

On the other hand, in step S16, in a case in which there is a region in which the reliability evaluation value is less than the evaluation value threshold, the processing proceeds to step S22.

In step S22, the adoption/rejection determination unit 44 determines the region in which the reliability evaluation value is less than the evaluation value threshold as the non-target region in which the strain is not used in a case in which the global strain is calculated. In addition, the adoption/rejection determination unit 44 determines the region in which the reliability evaluation value is equal to or greater than the evaluation value threshold as the target region used for calculating the global strain in a case in which the global strain is calculated.

In step S24, the display controller 36 notifies the operator by displaying the non-target region determined in step S22 on the display 14.

In step S26, the global strain calculation unit 40 determines whether or not the number of non-target regions determined in step S22 is equal to or greater than a predetermined number (two in the present embodiment). In a case in which the number of non-target regions is less than the predetermined number, the processing proceeds to step S20. In step S20, the global strain calculation unit 40 calculates the global strain by using the strain of the target region without using the strain of the non-target region. The display controller 36 displays the calculated global strain on the display 14.

In a case in which the number of non-target regions is equal to or greater than the predetermined number in step S26, the global strain calculation unit 40 ends the processing without calculating the global strain. Here, the display controller 36 may display that the global strain is not calculated on the display 14 to notify the operator.

The ultrasound tomographic image processing apparatus according to the present disclosure has been described above. However, the ultrasound tomographic image processing apparatus according to the present disclosure is not limited to the above-described embodiment, and various changes can be made without departing from the gist of the present disclosure.

For example, in the above-described embodiment, the ultrasound diagnostic apparatus 10 has each of the functions of the tracking processing unit 38, the global strain calculation unit 40, the reliability evaluation unit 42, and the adoption/rejection determination unit 44, but each of these functions may not necessarily be exhibited by the ultrasound diagnostic apparatus 10. For example, these functions may be exhibited by another apparatus such as a server. In this case, the other apparatus acquires the ultrasound tomographic image sequence representing the periodic motion of the target tissue from the ultrasound diagnostic apparatus or the like. In addition, all of the above-described functions may not be exhibited by one device, and the above-described functions may be exhibited by cooperation of a plurality of devices.

In the present embodiment, each processing is executed by any computer. In addition, any computer may execute these processes using a processor as hardware, a program as software, or a combination thereof. In such a case, the processor is configured to execute various types of processing in the present embodiment in cooperation with the program, and may function as each unit or each means in the present embodiment. Additionally, the execution order of the process by the processor is not limited to the order described above and may be changed as appropriate. Any computer may be a general purpose computer, a special purpose computer, a workstation, or other system capable of executing each process.

The processor may be configured with one or more pieces of hardware, and the type of hardware is not limited. For example, the processor may be configured using hardware such as a central processing unit (CPU), a micro-processing unit (MPU), a programmable logic device such as a field-programmable gate array (FPGA), a dedicated circuit for executing specific processing, such as an application-specific integrated circuit (ASIC), a graphics processing unit (GPU), or a neural processing unit (NPU). In addition, the types of hardware may be a combination of different types of hardware. In a case where a plurality of hardware components are configured to execute one or a plurality of processes of a certain processor, the plurality of hardware components may be present in physically separate devices or may be present within the same device. In addition, in any of the embodiments, the order of the processes performed by the processor is not limited to the order described above, and may be changed as appropriate. In addition, hardware is implemented in a form of an electric circuit (circuitry) in which circuit elements, such as semiconductor elements, are combined.

Further, the program may be software, such as firmware or a microcode. The program may also be, for example, a group of program modules, and each function may be implemented by a processor configured to execute the corresponding function. The program may be a program code or a plurality of code segments that are stored in one or a plurality of non-transitory computer-readable media (for example, storage media or other storages). The program may be divided and stored in a plurality of non-transitory computer-readable media present in devices physically separated from each other. The program code or the code segments may represent any combination of a procedure, a function, a subprogram, a routine, a subroutine, a module, a software package, a class, an instruction, a data structure, and a program statement. The program code or code segments may be connected to other code segments or to hardware circuits by transmitting and receiving information, data, arguments, parameters, or contents of memory.

In addition, the present invention can also be applied to a program and a program product.

What is claimed is:

1. An ultrasound tomographic image processing apparatus comprising:
a processor,
wherein the processor is configured to:
acquire an ultrasound tomographic image sequence that is formed by transmitting and receiving an ultrasonic wave to and from a target tissue of a subject and that is arranged in a time series representing a periodic motion of the target tissue;
calculate a deformation amount of the target tissue in the periodic motion for each of a plurality of regions of the target tissue defined in advance by executing tracking processing for tracking each part of the target tissue over time in the ultrasound tomographic image sequence;
calculate a reliability evaluation value representing reliability of the tracking processing for each of the regions;
determine regions in which the reliability evaluation value is less than a predetermined evaluation value threshold, as non-target regions in which the deformation amount is not used in a case of calculating an overall deformation amount that is a deformation amount of the target tissue as a whole in the periodic motion, based on the deformation amount for each of the regions; and
notify a user of the non-target regions.

2. The ultrasound tomographic image processing apparatus according to claim 1,
wherein the processor is configured to display, on a display unit, a target region that is a region in which the deformation amount is used in a case of calculating the overall deformation amount and the non-target region in a model diagram of the target tissue in which the plurality of regions are shown, in an identifiable manner.

3. The ultrasound tomographic image processing apparatus according to claim 1,
wherein the processor is configured to:
display at least one ultrasound tomographic image included in the ultrasound tomographic image sequence on a display unit;
display, on the display unit, a plurality of superimposed images that are superimposed on each of the regions in the ultrasound tomographic image to represent each of the regions and that correspond to each of the regions; and
display the superimposed image corresponding to the target region that is the region in which the deformation amount is used in a case of calculating the overall deformation amount and the superimposed image corresponding to the non-target region in an identifiable manner.

4. The ultrasound tomographic image processing apparatus according to claim 1,
wherein the processor is configured to display, on a display unit, a difference between the overall deformation amount calculated by using the deformation amount of each of the regions including the non-target region and the overall deformation amount calculated without using the deformation amount of the non-target region.

5. The ultrasound tomographic image processing apparatus according to claim 1,
wherein the processor is configured to notify the user that the overall deformation amount is not calculated in a case in which a number of non-target regions in the plurality of regions is equal to or greater than a predetermined number.

6. An ultrasound tomographic image processing apparatus comprising:
a processor,
wherein the processor is configured to:
acquire an ultrasound tomographic image sequence that is formed by transmitting and receiving an ultrasonic wave to and from a target tissue of a subject and that is arranged in a time series representing a periodic motion of the target tissue;
calculate a deformation amount of the target tissue in the periodic motion for each of a plurality of regions of the target tissue defined in advance by executing tracking processing for tracking each part of the target tissue over time in the ultrasound tomographic image sequence;
calculate a reliability evaluation value representing reliability of the tracking processing for each of the regions;
calculate an overall deformation amount that is a deformation amount of the target tissue as a whole in the periodic motion, based on the deformation amounts of regions in which the reliability evaluation value is equal to or greater than an evaluation value threshold, among the plurality of regions; and
display the overall deformation amount on a display unit in a case in which the overall deformation amount is calculated.

7. The ultrasound tomographic image processing apparatus according to claim 6,
wherein the processor is configured not to calculate the overall deformation amount in a case in which a number of the regions in which the reliability evaluation value is less than the evaluation value threshold is equal to or greater than a predetermined number, in the plurality of regions.

8. A non-transitory computer-readable storage medium storing an ultrasound tomographic image processing program causing a computer to execute a process comprising:
acquiring an ultrasound tomographic image sequence that is formed by transmitting and receiving an ultrasonic wave to and from a target tissue of a subject and that is arranged in a time series representing a periodic motion of the target tissue;
calculating a deformation amount of the target tissue in the periodic motion for each of a plurality of regions of the target tissue defined in advance by executing tracking processing for tracking each part of the target tissue over time in the ultrasound tomographic image sequence;
calculating a reliability evaluation value representing reliability of the tracking processing for each of the regions;
determining regions in which the reliability evaluation value is less than a predetermined evaluation value threshold, as non-target regions in which the deformation amount is not used in a case of calculating an overall deformation amount that is a deformation amount of the target tissue as a whole in the periodic motion, based on the deformation amount for each of the regions; and
notifying a user of the non-target regions.
